(19) United States
(12) Patent Application Publication
(10) Pub. No.: US 2018/0320215 A1
(43) Pub. Date: Nov. 8, 2018

(54) METHOD FOR SCREENING FOR ANTIMICROBIAL AGENT

(71) Applicant: Hyundai Motor Company, Seoul (KR)

(72) Inventors: So Yoon Park, Suwon (KR); Tae Hee Lee, Hwaseong (KR); Ji Wan Kim, Hwaseong (KR); Ki Young Yoon, Seoul (KR)

(21) Appl. No.: 16/022,128

(22) Filed: Jun. 28, 2018

Related U.S. Application Data
(63) Continuation of application No. PCT/KR2016/015500, filed on Dec. 29, 2016.

(30) Foreign Application Priority Data
Dec. 29, 2015 (KR) 10-2015-0188631
Dec. 29, 2015 (KR) 10-2015-0188643
Dec. 29, 2015 (KR) 10-2015-0188657
Dec. 29, 2015 (KR) 10-2015-0188667
Dec. 29, 2015 (KR) 10-2015-0188675
Dec. 29, 2015 (KR) 10-2015-0188680

(51) Int. Cl. C12Q 1/18; A61L 9/01; C12Q 1/689

(52) U.S. Cl. CPC C12Q 1/18; B60H 3/00; C12Q 1/689; A61L 9/01

(57) ABSTRACT

Disclosed is a method for screening for an antimicrobial agent capable of controlling microorganisms generating offensive odor in an air conditioning apparatus. Further disclosed is a method for removing offensive odor in the air conditioning apparatus. The microorganism causing offensive odor in the air conditioning apparatus may be used in the development of a novel antimicrobial agent or in the development of an aromatic for blocking offensive odor by establishing the chemical properties of metabolites of the microorganisms. In addition, there are various industrial applications, for example, by fundamentally removing the cause of offensive odor by creating an environment in which the microorganisms cannot live in the air conditioning apparatus in advance.

### CROSS REFERENCES TO RELATED APPLICATIONS

This Application is a Continuation of Application PCT/KR2016/015500 filed on Dec. 29, 2016 which claims priority from Application 10-2015-0188643 filed on Dec. 29, 2015 in the Republic of Korea; which claims priority from Application 10-2015-0188631 filed on Dec. 29, 2015 in the Republic of Korea; which claims priority from Application 10-2015-0188680 filed on Dec. 29, 2015 in the Republic of Korea; which claims priority from Application 10-2015-0188675 filed on Dec. 29, 2015 in the Republic of Korea; which claims priority from Application 10-2015-0188667 filed on Dec. 29, 2015 in the Republic of Korea; which claims priority from Application 10-2015-0188657 filed on Dec. 29, 2015 in the Republic of Korea. The entire contents of these applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 24, 2018, is named 058268-823C01US_ST25.txt and is 781 bytes in size.

### TECHNICAL FIELD

The present disclosure relates to a method of screening an antimicrobial agent to control odor-causing microorganisms in an air conditioning system and a method for removing odors in an air conditioning system.

### BACKGROUND

Clean air is recognized as essential to human health and well-being, and offensive odors or contaminated air may disrupt the pleasant environment. For example, quality of unsatisfactory indoor air under closed conditions may be determined by the following factors: indoor air contamination that is generated directly from the material constituting the enclosed environment (building, vehicle, and the like) and air pollution caused by human activity or a substance introduced from the outside.

Air conditioning systems are systems that reduce the indoor temperature and optimize the indoor environment, for air conditioning including conditioning the temperature, humidity, airflow and cleanliness of the air in buildings, vehicles, trains, ships, aircraft, and the like. These air conditioning systems have been used increasingly with improvement in standards of living. However, although the air conditioning systems has brought about a great development in basic functions, many environmental issues to improve the quality of indoor air remain unsolved.

Although the cause of odor of air conditioners among air conditioning systems is known to be metabolites of fungi and bacteria, there is no specific report regarding the types of fungi and bacteria, and amounts of metabolites secreted by these microorganisms.

The air conditioning system may have a structure where all air passing through the blower passes through the evaporator core (eva core). When heat exchange is carried out between cold refrigerant and air, water may condense on the surface of the evaporator core due to temperature difference. The continuous condensation of condensate water provides the environment for the growth or proliferation of fungi and bacteria. When fungi and bacteria proliferate in the evaporator core exposed to outside air, volatile organic compounds (mVOCs) of microorganisms may be produced from metabolites of bacteria perforated on the surface of the evaporator core. When the air passing through the evaporator core is blown into the room, the room may be exposed to odor of fungi and bacteria upon use for a long time due to volatile organic compounds produced by microorganisms.

The surface of the evaporator core where odors are emitted may be covered with a biofilm as the air conditioning system is used for a long period of time. For example, the biofilms are composed of bacteria, cell clusters and extracellular polymeric substance (EPS). EPS contains a variety of ingredients including proteins, polysaccharides, polyuronic acid, nucleic acids, lipids and the like. On the surface of the evaporator core, a variety of bacteria and fungi proliferate using biofilms as nutrients to release organic compounds (mVOCs) as metabolites by microorganisms. At this time, the odor emitted from the organic compounds (mVOCs) may produce offensive odor of air conditioners.

Various types of fragrances may be used to remove offensive odors are commercially available, but such fragrances cannot fundamentally remove fungi and bacteria growing on the evaporator core, and merely serve to temporarily relieve unpleasant odors. Currently commercially available antimicrobial agents have been used against common pathogens, but specific antimicrobial agents have not been developed to target specific fungi or bacteria in the air conditioning system.

Accordingly, there is an urgent need to develop antimicrobial agents for a pleasant indoor air environment and technologies for removing unpleasant odors using the antimicrobial agents by clearly identifying the kinds of fungi and bacteria grown on the evaporator core and specifically blocking or preventing propagation of fungi and bacteria.

The above description of the background art is intended only to improve understanding of the background of the present disclosure and should not be construed as recognizing that the above-described technologies are known to those having ordinary skill in the technical field to which the present disclosure pertains.

### SUMMARY OF THE INVENTION

In preferred aspects, the present disclosure provides methods of identifying and effectively controlling odor-generating microorganisms in an air conditioning system. For example, six species of microorganisms which generate odors and form a biofilm in the air conditioning system were isolated. Accordingly, when controlling growth of these microorganisms or a combination thereof, an offensive odor generated in an air conditioning system can be prevented.

In one aspect of the present disclosure, provided is a method of screening an antimicrobial agent against microorganisms in an air-conditioning system. The microorganism may be at least one microorganism causing an offensive odor in an air-conditioning system. Preferably, the microorganism may include at least one selected from the group consisting of Pelomonas puraquae, Spirosoma radiotolerans, Fibrella aestuarina, Chryseobacterium geocarposphaerae, Spirosoma linguale, and Geobacillus toebii.

The method may include steps of: (a) preparing microorganism or a culture solution thereof; (b) contacting the microorganism or a culture solution thereof with a sample including the antimicrobial agent; (c) measuring growth of the microorganism; and (d) determining whether the sample has antibacterial activity to reduce odors in an air-conditioning system when growth of the microorganism is inhibited.

Preferably, the air-conditioning system may be an air conditioner. The microorganism may form a biofilm in an evaporator core in the air-conditioning system to induce odors. A material for the evaporator core may suitably include aluminum, an aluminum alloy, copper or a copper alloy.

As used herein, the term “antimicrobial” refers to a property of a substance (e.g., a compound or a composition) that can effect a parameter of a microorganism, including death, eradication, elimination, reduction in number, reduction of growth rate, inhibition of growth, change in population distribution of one or more species of microbial life forms. This term encompasses antibacterial agents and antibiotics.

An “antimicrobial agent”, as used herein, refers to an agent that is capable of decreasing or eliminating or inhibiting the growth of microorganisms such as that term is known in the art (exemplary microorganisms include microbes such as bacteria, fungi, viruses and other pathogens).

The term “biofilm” as used herein refers to an aggregate of bacterial microorganisms in which bacterial cells adhere to each other and/or to a surface. These adherent cells are often covered with a matrix of extracellular polymeric substance (EPS), which is produced by the cells and/or host. Biofilm EPS has been characterized as composed of extracellular DNA, proteins, and polysaccharides. Such biofilms may form on any living or non-living surfaces, in particular both on solid surfaces as colonies and/or on liquid surfaces as pellicles.

Preferably, the Pelomonas puraquae may be Pelomonas puraquae HKMC-113 (accession number: KCCM11689P), the Spirosoma radiotolerans may be Spirosoma radiotolerans HKMC-114 (accession number: KCCM11690P), the Fibrella aestuarina may be Fibrella aestuarina HKMC-115 (accession number: KCCM11691P), the Chryseobacterium geocarposphaerae may be Chryseobacterium geocarposphaerae HKMC-116 (accession number: KCCM11692P), the Spirosoma linguale is Spirosoma linguale HKMC-117 (accession number: KCCM11693P), and/or the Geobacillus toebii may be Geobacillus toebii HKMC-118 (accession number: KCCM11694P).

Further provided is an antimicrobial agent screened by the method as described herein.

Still further provided is a kit including the antimicrobial agent as described herein. In another aspect of the present disclosure, provided are odor-generating microorganisms in an air-conditioning system.

In another aspect, provided is a method of inhibiting growth of odor-generating microorganisms in an air-conditioning system. The method may include coating or spraying an antimicrobial agent screened by the method as described herein on the air-conditioning system.

In another aspect, provided is a method of removing offensive odors in an air-conditioning system. The method may include isolating or removing at least one odor-generating microorganisms from the air-conditioning system. The odor-generating microorganism may be selected from the group consisting of Pelomonas puraquae, Spirosoma radiotolerans, Fibrella aestuarina, Chryseobacterium geocarposphaerae, Spirosoma linguale, and Geobacillus toebii.

In another aspect, provided is a method of removing offensive odors in an air-conditioning system. The method may include inhibiting growth of odorgenerating microorganisms in the air-conditioning system. The odor-generating microorganism may be selected from the group consisting of Pelomonas puraquae, Spirosoma radiotolerans, Fibrella aestuarina, Chryseobacterium geocarposphaerae, Spirosoma linguale, and Geobacillus toebii.

Other features and aspects of the present disclosure will be apparent from the following detailed description, drawings and claims.

### BRIEF DESCRIPTION OF THE DRAWING

FIG. 1 is an image showing an exemplary specimen sampled from an evaporator core in an odor-causing secondhand vehicle;

FIG. 2 is an image showing an exemplary method of testing antibacterial activity according to an exemplary embodiment of the present disclosure; and

FIG. 3 is an image showing culturing combinations of dominant odorless microorganisms using an aluminum fin which is a material for an evaporator core.

### DETAILED DESCRIPTION

The terminology used herein is for the purpose of describing particular embodiments only and is not intended to be limiting. As used herein, the singular forms “a,” “an” and “the” are intended to include the plural forms as well, unless the context clearly indicates otherwise. It will be further understood that the terms “comprise”, “include”, “have”, etc. when used in this specification, specify the presence of stated features, regions, integers, steps, operations, elements and/or components but do not preclude the presence or addition of one or more other features, regions, integers, steps, operations, elements, components, and/or combinations thereof.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Further, unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless otherwise defined, all terms including technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Hereinafter, a detailed description will be given according to various exemplary embodiments of the present disclosure with reference to the appended drawings.

In one aspect, the present disclosure provides a method of screening for antimicrobial agents to reduce offensive odors in an air-conditioning system. The method may be a method of screening an antimicrobial agent in an air-conditioning system to remove odors generated from at least one microorganism selected from the group consisting of Pelomonas puraquae, Spirosoma radiotolerans, Fibrella aestuarina, Chryseobacterium geocarposphaerae, Spirosoma linguale, and Geobacillus toebii. The method may include: (a) preparing the microorganism or a culture solution thereof; (b) contacting the microorganism or a culture solution thereof with a sample including the antimicrobial agent; (c) measuring growth inhibition of the microorganism; and (d) determining whether the sample has antibacterial activity to reduce offensive odors in an air-conditioning system, when growth of the microorganism is inhibited.

The present inventors made attempts to identify microorganisms generating offensive odors and found methods which are capable of effectively controlling microorganisms. For example, they successfully isolated six species of microorganisms, which created and grew a biofilm in an air conditioning system, and found that offensive odors generated from the air conditioning system can be significantly prevented by controlling these microorganisms.

As herein used, the term “air conditioning system” generically refers to a system which can maintain the temperature, humidity, cleanliness, flow or the like of air pleasant in an area, a part or entirety of which is isolated from an outdoor environment. Preferably, for example, the isolated area may be an indoor area, a part or the entirety of which is isolated from an outdoor environment, like the inside of a building or the inside of a vehicle, train, ship, aircraft or the like. Preferably, the air conditioning system is for example an air conditioner.

Based on the structure of the air conditioning system, all air having passed through a blower passes through the evaporator core and water continuously condenses on the surface of the evaporator core due to temperature difference, providing an environment which is optimal for growth of microorganisms. After a long time, a biofilm may be formed. The microorganisms in the evaporator may metabolize various indoor and outdoor materials as nutrients present in the air, generating odors derived from volatile organic compounds (mVOCs) produced as a result of metabolism.

Biofilms are a form of microbial communities wherein microorganisms live as clusters, have a structure in which a layer is surrounded by one membrane, serve to protect microorganisms from the outside environment and provide nutrients. Exopolymeric substances (EPSs) may be present as an ingredient constituting the film and contain a variety of ingredients such as proteins, polysaccharides, polyuronic acids, nucleic acids, and lipids. On the surface of the evaporator core, various microorganisms may proliferate from the substances as nutrients and emit unpleasant odors from metabolites.

The present inventors isolated microorganisms which generate odors from the evaporator core and, as a result of culture of the microorganisms, separated and cultured dominant strains among microorganisms forming colonies. The method of separating and culturing dominant strains may be carried out using a variety of methods well-known to those skilled in the art. For example, dominant microorganisms can be selected through morphological approaches, such as dilution rate, and color, size or shape of colonies.

The isolated microorganism may include the genera Fibrella, Chryseobacterium, Spirosoma, Geobacillus, or Pelomonas, preferably, Fibrella aestuarina, Chryseobacterium geocarposphaerae, Spirosoma linguale, Spirosoma radiotolerans, Geobacillus toebii, or Pelomonas puraquae.

The microorganisms were deposited at the Korea Culture Center of Microorganisms on Apr. 17, 2015 and were given the following accession numbers: Fibrella aestuarina HKMC-115 (accession number: KCCM 11691P), Chryseobacterium geocarposphaerae HKMC-116 (accession number: KCCM 11692P), Spirosoma linguale HKMC-117 (accession number: KCCM 11693P), Spirosoma radiotolerans HKMC-114 (accession number: KCCM 11690P), Geobacillus toebii HKMC-118 (accession number: KCCM 11694P), and Pelomonas puraquae HKMC-113 (accession number: KCCM 11689P).

The odor-generating microorganisms have a variety of industrial applicability. For example, the odor-causing microorganisms may be used to screen and/or develop novel antibacterial agents to inhibit growth of microorganisms and develop an air freshener for removing offensive odors by identifying the chemical properties of the metabolites of the microorganisms. In addition, the odor-causing microorganisms may be used to fundamentally remove the cause of offensive odors by providing an air-conditioning system with an environment where the microorganisms cannot live.

The sample used in the method for screening an antimicrobial agent of the present disclosure may be used to determine whether it has antimicrobial activity against the microorganisms. For example, when a particular sample has antimicrobial activity against Pelomonas puraquae, the sample may be screened and identified as an antimicrobial agent against Pelomonas puraquae.

Preferably, the antimicrobial agent screened by the screening method of the present disclosure may have antimicrobial activity against Pelomonas puraquae, more preferably, against other species of microorganisms.

For example, some antimicrobial agents may have antimicrobial activity against all six species of microorganisms and another antimicrobial agent may have no antimicrobial activity at all against one or more of the species. In addition, the antimicrobial agent having antimicrobial activity against all six species of microorganisms may have different antimicrobial activity against different microorganisms (see TABLE 8).

In one preferred exemplary embodiment of the present disclosure, the sample to be screened may include a single compound, a mixture of compounds, an animal or plant extract, a biological agent containing genetic information such as a nucleotide, a polypeptide and the like, and a mixture of compound and biological agent.

In another aspect, the present disclosure provides a microorganism causing offensive odors in an air-conditioning system.

Preferably, the microorganism causing an offensive odor may include one or more selected from the group consisting of Pelomonas puraquae, Spirosoma radiotolerans, Fibrella aestuarina, Chryseobacterium geocarposphaerae, Spirosoma linguale, and Geobacillus toebii.

In another aspect, the present disclosure provides a method for inhibiting the growth of a microorganism causing offensive odors in an air-conditioning system. The method may include coating or spraying the antimicrobial agent onto an air-conditioning system.

The antimicrobial agent may be any antimicrobial agent which is determined or can be determined to have antimicrobial activity against one or more microorganisms selected from the group consisting of Pelomonas puraquae, Spirosoma radiotolerans, Fibrella aestuarina, Chryseobacterium geocarposphaerae, Spirosoma linguale, and Geobacillus toebii.

The antimicrobial agent may be coated or sprayed into an air-conditioning system to inhibit growth of the odor-causing microorganisms and microorganisms including the same, and the coating or spraying may be carried out in various forms known in the art, such as gas, liquid, gel or suspension of a solid.

In addition, the coating or spraying may be performed partly or wholly on the inner surface or inner components of the air-conditioning system. Preferably, the coating or spraying may be performed on an evaporator core in the air-conditioning system. The inhibition of growth may be carried out by applying, coating or spraying the antimicrobial agent after the odor-generating microorganisms form a biofilm or by applying or spraying antimicrobial agent to prevent growth of the microorganisms before the odor-generating microorganisms form a biofilm.

In another aspect, the present disclosure provides a method for removing an offensive odor in an air-conditioning system. The method may include isolating or removing a microorganism causing offensive odors from the air-conditioning system.

The removal of offensive odors may include all or some of offensive odors, and the coating or spraying may be performed to prevent offensive odors before the offensive odors are generated.

Various microorganisms proliferate in an air-conditioning system. These microorganisms may be broadly classified into microorganisms causing offensive odors and microorganisms not causing offensive odors. Accordingly, when the antimicrobial agent acts specifically only on the microorganisms causing offensive odors or has inhibitory activity against the growth of all or some of the dominant species of microorganisms causing offensive odors, the offensive odors of the air-conditioning system may be partially or completely removed or improved.

In another aspect, the present disclosure provides a method for removing offensive odors in an air-conditioning system. The method may include isolating or removing microorganisms causing offensive odors from an air-conditioning system.

The microorganisms described above or microorganisms including the same may be partially or completely isolated or removed via a physical, chemical or biological method. The physical method may be one of artificially isolating or removing the aforementioned microorganism or a microorganism including at least one of the same using a physical apparatus. The chemical method may be one of isolating or removing the aforementioned microorganism or a microorganism including at least one of the same using an antimicrobial agent or a sterilizer against the microorganism. The biological method may be one of isolating or removing the microorganisms using a biological agent which is toxic to the microorganisms or using another microorganism which competes with the microorganism for survival. However, the present disclosure is not limited by these examples.

In another aspect, the present disclosure provides a method for removing offensive odors in an air conditioning system. The method may include inhibiting growth of odor-causing microorganisms in the air conditioning system.

The present disclosure provides a microorganism causing offensive odors in an air-conditioning system. In addition, the present disclosure provides a method for screening for an antibacterial agent to control microorganisms. Furthermore, the present disclosure provides a method for removing offensive odors in an air-conditioning system by controlling the microorganisms.

The odor-causing microorganisms in the air-conditioning system may be used to develop novel antibacterial agents or to develop an air fresher to block offensive odors by identifying the chemical properties of metabolites of microorganisms. In addition, the odor-causing microorganisms have various industrial applicability of fundamentally removing the cause of odors by previously creating an environment to prevent growth of the microorganisms in the air-conditioning system.

### EXAMPLES

Hereinafter, the present disclosure will be described in more detail with reference to examples. These examples are provided only for illustration of the present disclosure and it would be obvious to those skilled in the art that the scope of the present disclosure is not limited by the examples depending on the subject matter of the present disclosure.

### Example 1: Selection of Odor-Causing Dominant Microorganisms

1. Obtaining Odorous Vehicles and Separation of Air-Conditioning System

In order to identify the cause of offensive odors generated in a closed environment, like the inside of a vehicle, the present inventors secured ten kinds of second-hand vehicles generating offensive odors depending on season (winter: February to March, summer: June to July), isolated an air-conditioning system mounted on the respective vehicles, and detached an evaporator core where a biofilm was predicted to be formed by odor-causing microorganisms to conduct specimen sampling (TABLE 1).

TABLE 1

| No. | Vehicle mileage | | Season |
|---|---|---|---|
| 1 | 89,000 | km | Winter (February-March) |
| 2 | 70,000 | km | |
| 3 | 10,300 | km | |
| 4 | 37,100 | km | |
| 5 | 149,970 | km | |
| 6 | 35,000 | km | Summer (June-July) |
| 7 | 28,000 | km | |
| 8 | 42,000 | km | |
| 9 | 110,000 | km | |
| 10 | 90,000 | km | |

2. Evaporator Core Specimen Sampling

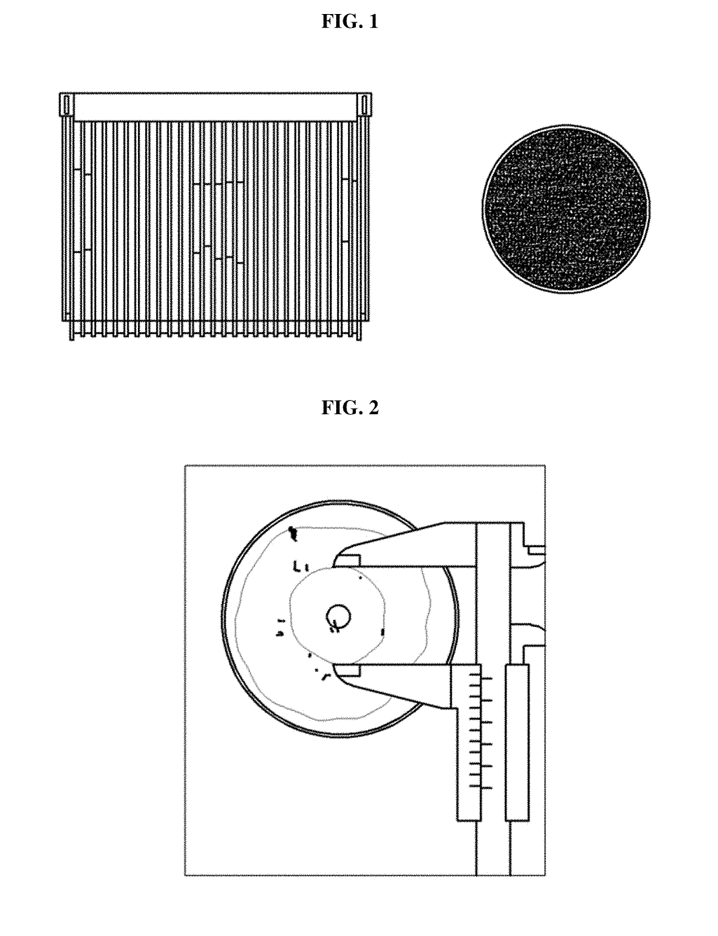

The evaporator core samples obtained from odorous second-hand vehicles 1 to 10 were sealed in a polyethylene bag and refrigerated at a temperature of 4° C. before use. In order to isolate and culture microorganisms, 5 g of specimens were collected from any spots including front and back parts in respective evaporator cores using sterilized long nose pliers and then mixed before use (FIG. 1).

3. Separation of Microorganisms

The microorganisms were separated from specimens acquired from the evaporator cores in accordance with the following process.

\textcircled{1} Specimens extracted from the evaporator cores were mixed and fed into a stirrer.

\textcircled{2} Sterilized 1× phosphate buffed saline (PBS) was fed into a 200 ml stirrer.

\textcircled{3} The mixed specimen was stirred with PBS for 30 seconds.

\textcircled{4} The stirrer was placed on ice for one minute.

\textcircled{5} Steps \textcircled{3} and \textcircled{4} were repeated twice.

\textcircled{6} The suspension was centrifuged at a temperature of 4° C. and 13,000 rpm for 3 minutes.

\textcircled{7} Only the supernatant was collected and transferred to a new tube.

\textcircled{8} The sterilized swab was soaked with the supernatant and the surface of the evaporator core, from which the sample was collected, was cleaned with the swab several times.

\textcircled{9} Only the head of the cleaned swab was immersed in the supernatant and vortexing was conducted.

\textcircled{10} The precipitate obtained in step \textcircled{6} was mixed with the mixture of step \textcircled{9} and the resulting mixture was used as an inoculation stock.

After steps \textcircled{1} to \textcircled{10}, microorganisms were separated by physical detachment from evaporator cores mounted on vehicle models 1 to 10.

4. Separation of Odor-Causing Microorganisms and Selection of Dominant Strains

The separation of bacteria from the air conditioner is generally carried out by performing heterotrophic plate culture on aerobic heterotrophic bacteria which are called general bacteria. Separation of bacteria is carried out at a temperature of 28 to 30° C. for 14 days using two complex nutrient media including PTYG agar medium and R2A agar medium. In the case of PTYG agar medium, peptone 0.25 g (Difco), triptone 0.25 g (Difco), yeast extract 0.5 g (Difco), glucose 0.5 g (Difco), MgSO₄ 30 mg (Sigma), CaCl₂ 3 mg (Sigma), and Bactoagar 15 g (Difco) were added to 980 ml of distilled water, pH was adjusted to 7.0 and the resulting mixture was autoclaved at 121° C. for 15 minutes. In the case of R2A agar medium, yeast extract 0.5 g (Difco), proteose peptone No. 3 0.5 g (Difco), casamino acids 0.5 g (Difco), dextrose 0.5 g (Difco), soluble starch 0.5 g (Difco), sodium pyruvate 0.3 g (Difco), dipotassium sulfate 0.3 g (Difco), magnesium sulfate 0.05 g (Difco) and bacto agar 15 g (Difco) were added to distilled water 980 ml, pH was adjusted to 7.2 (final: 1,000 ml) and the resulting mixture was autoclaved at a temperature of 121° C. for 15 minutes. In order to isolate non-dominant bacteria, kanamycin, ampicillin and chloramphenicol were added at a concentration of 100 ppm when the medium temperature reached a temperature of 50° C. after sterilization of the filter, to produce antibiotic media.

To separately culture dominant strains, first, various dominant strains should be selected through morphological approach of dilution ratio, and color, size and shape of colonies and the like.

\textcircled{1} Fungi and bacteria were separately isolated from the separately cultured media.

\textcircled{2} A variety of bacteria having different morphologies were inoculated into a complex medium using a loop and purely isolated.

\textcircled{3} The medium growing the best was selected from inoculated media and passage culture was conducted.

\textcircled{4} The end of the mycelium of fungi was isolated with a scalpel and inoculated into a complex medium.

\textcircled{5} In the case of fungi strains as well, the medium growing the most was selected from inoculated media and passage culture was conducted.

5. Identification of Dominant Strains

In order to accurately identify the isolated microorganisms, 16s rRNA identification including the following steps was conducted.

a) Investigation of Fingerprints Through REP-PCR Pattern Analysis

REP-PCR is a molecular biological method of analyzing the structure of bacterial chromosomes and is a fingerprinting method which is capable of distinguishing specific bacterial strains from other bacteria. Genetic characteristics were analyzed in accordance with respective processes to conduct REP-PCR.

(1) Cell Lysis Process

\textcircled{1} 2.5 μl of Lyse-N-Go PCR Reagent (Thermo) was placed in a PCR tube.

\textcircled{2} Colonies were harvested with a pipette at a clean bench, placed in the tube and pipetting was conducted. The amount of colonies harvested should be determined not to make the solution slightly hazy.

\textcircled{3} According to manufacturer's instructions, culture was conducted in a PCR machine.

(2) PCR Reaction

Suitable amounts of ingredients required for PCR reaction described in the following TABLE 2 were mixed to prepare a reaction mixture and, as shown in TABLE 3, pre-denaturation at a temperature of 93° C. for 7 minutes, denaturation at a temperature of 92° C. for 1 minute, annealing at a temperature of 51.5° C. for 1 minute, and extension at a temperature of 65° C. for 8 minutes were conducted, and denaturation, annealing and extension processes were repeated 33 times to conduct PCR amplification.

TABLE 2

| | | |
|---|---|---|
| \textcircled{1} dNTP (2.5 mM each) | 12.5 | μl |
| \textcircled{2} Gitschier buffer | 5.0 | μl |
| \textcircled{3} DMSO (100%) | 2.5 | μl |
| \textcircled{4} Autoclaved 3°D.W. | 0.3 | μl |
| \textcircled{5} BOXA1R primer(50 pmole/μl) | 1.0 | μl |
| 5′-CTACGGCAAGGCGACGCTGACG-3′ (SEQ ID NO: 1) | | |
| \textcircled{6} BSA (10 mg/ml) | 0.4 | μl |
| \textcircled{7} Bacterial DNA | 2.5 | μl |
| \textcircled{8} Taq polymerase(Roche) (5 U/μl) | 0.8 | μl |

TABLE 3

| | | | | |
|---|---|---|---|---|
| step 1 | 93° | C. | 7 | min |
| step 2 | 92° | C. | 1 | min |
| step 3 | 51.5° | C. | 1 | min |
| step 4 | 65° | C. | 8 | min |

step 2, 3, 4: additional 33 cycles

| | | | | |
|---|---|---|---|---|
| step 6 | 65° | C. | 16 | min |
| step 7 | 4° | C. | | |

(3) Gel Electrophoresis

The DNA fragments amplified by PCR were collected, 1.2-1.5% agarose gel supplemented with EtBr was used, and a mixture of 6× dye and a sample in a ratio of 1 to 5 was loaded in an amount as much as possible. Since most PCR products were between 100 and 1,000 bp, they were loaded with a 100 bp ladder, and electrophoresis was conducted as slow as possible such that the middle (50 V) of bromophenol blue and xylene cyanol dyes reached the middle of the entire gel. Strains that have identical DNA patterns on gel are considered to be the same strains.

(b) Identification of Air Conditioner Dominant Bacteria Through 16S rRNA Genetic Analysis

16S rRNA (ribosomal ribonucleic acid) genes are used for identification of genetic classes of bacteria and can be identified at the level of genus and species of bacteria classified by REP-PCR.

(1) Cell Lysis Process

\textcircled{1} 5 μl of Lyse-N-Go PCR Reagent (Thermo) was placed in a PCR tube.

\textcircled{2} Colonies were harvested with a pipette at a clean bench, placed in the tube and pipetting was conducted. The amount of colonies harvested should be determined not to make the solution slightly hazy.

\textcircled{3} According to manufacturer's instructions, culture was conducted in a PCR machine (TABLE 4).

TABLE 4

| Cycle | Temperature(° C.) | Time(seconds) |
|---|---|---|
| 1 | 65 | 30 |
| 2 | 8 | 30 |
| 3 | 65 | 90 |
| 4 | 97 | 180 |
| 5 | 8 | 60 |
| 6 | 65 | 180 |

(2) 16S rRNA Genetic PCR

PCR conditions (Total 50 μl): ingredients for the solution excluding DNAs and Taq were mixed in predetermined amounts as shown in the following TABLE 5 and the resulting mixture was added to 44.5 μl of a lysis solution. Then, as shown in the following TABLE 6, pre-denaturation at a temperature of 94° C. for 5 minutes, denaturation at a temperature of 94° C. for 1 minute, annealing at a temperature of 55° C. for 1 minute, and extension at a temperature of 72° C. for 1 min 30 seconds were conducted, and denaturation, annealing and extension steps were conducted 29 times to perform PCR amplification.

TABLE 5

| | | |
|---|---|---|
| Autoclaved 3°D.W. | 22 | μl |
| 10xbuffer (Roche) | 5 | μl |
| dNTP (Roche, 2.5 mM) | 5 | μl |
| DMSO | 5 | μl |
| BSA (10 mg/ml) | 2.5 | μl |
| 27 mf (20 pmole/μl) | 2.5 | μl |
| 1492r (20 pmole/μl) | 2.5 | μl |
| DNA | 5 | μl |
| Taq (Roche) | 0.5 | μl |

TABLE 6

| | | | | |
|---|---|---|---|---|
| step 1 | 94° | C. | 5 | min |
| step 2 | 94° | C. | 1 | min |
| step 3 | 55° | C. | 1 | min |
| step 4 | 72° | C. | 1 | min 30 sec |

Go to step 2: additional 29 cycles

| | | | | |
|---|---|---|---|---|
| step 6 | 72° | C. | 10 | min |

| | | | |
|---|---|---|---|
| step 7 | 4° | C. | hold |

(3) PCR Purification

The products amplified by 16S-rRNA genetic PCR were purified using a QIAQUICK PCR purification kit (Qiagen) in accordance with the following process.

\textcircled{1} 5× volume of PB buffer of the PCR product was added.

\textcircled{2} The mixed solution was seeded on a QIAquick column.

\textcircled{3} For binding of DNAs, centrifugation was conducted for one minute and the supernatant was removed.

\textcircled{4} For washing, 750 μl of PE buffer was placed in a QIAQUICK column, centrifugation was conducted for one minute and the supernatant was removed.

\textcircled{5} Centrifugation was conducted again for one minute.

\textcircled{6} The QIAQUICK column was transferred to a new tube.

\textcircled{7} In order to extract DNAs, 30 μl of EB buffer was added thereto and was allowed to stand for one minute.

\textcircled{8} Centrifugation was conducted for one minute to allow DNAs dissolved in the EB buffer to be collected in the tube.

As a result of the test, in order to check whether or not purely isolated microorganisms generate odors, purely isolated microorganisms were cultured by the following method and sensory evaluation was conducted.

\textcircled{1} Pure separately cultured microorganisms were inoculated into a liquid nutrient medium.

\textcircled{2} The inoculated medium was cultured at a temperature of 28° C. for 5 to 7 days.

\textcircled{3} 100 μl of the bacteria cultured in the liquid medium was inoculated into a solid nutrient medium.

\textcircled{4} The inoculated bacteria was evenly spread using a spreader.

\textcircled{5} A petri dish was sealed and cultured at a temperature of 28° C. for 10 days.

Sensory evaluation was conducted, based on a 5-grade method using seven panels, odor-causing microorganisms were selected using the average after evaluation of odor intensity, six dominant strains were identified through identification of the 16S rRNA genetic analysis, and these dominant strains were deposited at the Korea Culture Center of Microorganisms on Apr. 17, 2015.

TABLE 7

| | | | Accession |
|---|---|---|---|
| No. | Identification No. | Name of microorganism | No. |
| 1 | HKMC-113 | Pelomonas puraquae | KCCM |
| | | | 11689P |
| 2 | HKMC-114 | Spirosoma radiotolerans | KCCM |
| | | | 11690P |
| 3 | HKMC-115 | Fibrella aestuarina | KCCM |
| | | | 11691P |
| 4 | HKMC-116 | Chryseobacterium | KCCM |
| | | geocarposphaerae | 11692P |
| 5 | HKMC-117 | Spirosoma linguale | KCCM |
| | | | 11693P |
| 6 | HKMC-118 | Geobacillus toebii | KCCM |
| | | | 11694P |

### Example 2: Evaluation of Antibacterial Activity of Selected Odor-Causing Microorganisms Depending on Antibacterial Agent

1. Test Process

The present inventors evaluated antibacterial activity of dominant microorganisms selected in Example 1 using a variety of commercially available antibacterial agents. The antibacterial agents used in the present disclosure are given below:

Antibacterial agent A: Kimcare (Yuhan Kimberly, Ltd.)

Antibacterial agent B: Febreze (P&G)

Antibacterial agent C: mass-produced antibacterial agent containing methyl alcohol 45-50%, chromium sulfate (CAS 10101-53-8) 1-5%, bromine 1-5% and water

The evaluation of antibacterial activity was conducted by the following steps:

\textcircled{1} Preparing sterilized filter paper

\textcircled{2} Preparing three kinds of antibacterial agents (control group: group not-treated with antibacterial agent, test groups: antibacterial agent A, antibacterial agent B, antibacterial agent C)

\textcircled{3} Feeding filter paper to antibacterial agent

\textcircled{4} coating respective odor-causing microorganisms in a nutrient medium

\textcircled{5} placing antibacterial agent-containing filter papers in odor-causing microorganisms-coated nutrient medium

\textcircled{6} Culturing at a temperature of 28 to 30° C. for 5 days

\textcircled{7} Measuring an area of growth inhibition.

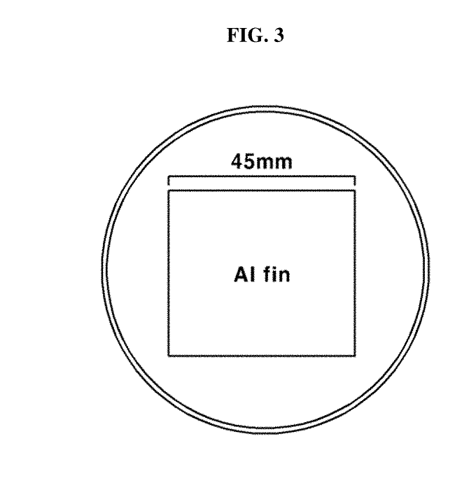

Measurement of the area of growth inhibition was carried out by measuring the diameter of the area of growth inhibition using Vernier calipers, and the method is shown in detail in FIG. 2.

2. Test Result

An average of three diameters of the area of growth inhibition obtained by testing each of six strains of odor-causing microorganisms three times is shown in TABLE 8.

TABLE 8

| | | No anti- | | | |
|---|---|---|---|---|---|
| | Name of microorganism | bacterial | | | |
| No. | (Deposition name) | Agent | A | B | C |
| 1 | Pelomonas puraquae | 0 | 1.17 | 2.07 | 3.93 |
| | HKMC-113 | | | | |
| 2 | Spirosoma radiotolerans | 0 | 2.03 | 2.17 | 4.57 |
| | HKMC-114 | | | | |
| 3 | Fibrella aestuarina | 0 | 2.13 | 2.63 | 4.30 |
| | HKMC-115 | | | | |
| 4 | Chryseobacterium | 0 | 0.97 | 1.57 | 2.70 |
| | geocarposphaerae | | | | |
| | HKMC-116 | | | | |
| 5 | Spirosoma linguale | 0 | 1.83 | 2.10 | 4.83 |
| | HKMC-117 | | | | |
| 6 | Geobacillus toebii | 0 | 1.73 | 1.93 | 2.20 |
| | strain R-35642HKMC-118 | | | | |

(unit: cm)

As shown in TABLE 8, antibacterial agent A exhibited weaker antibacterial activity against Chryseobacterium geocarposphaerae, and antibacterial agent B exhibits strong antibacterial activity against Fibrella aestuarina, but weaker antibacterial activity against Chryseobacterium geocarposphaerae.

In addition, antibacterial agent C exhibited weak antibacterial activity against Geobacillus toebii, but exhibited stronger overall antibacterial activity against six strains of microorganisms than antibacterial agents A and B.

### Example 3: Evaluation of Odors of Evaporator Core from which Odor-Causing Microorganisms are Removed

In order to reproduce the evaporator core from which odorous microorganisms were removed or separated, the present inventors cultured a combination of odorless microorganisms excluding the odorous microorganism of Example 1, among dominant microorganisms grown in the evaporator core, using an aluminum fin which is a material for the evaporator core (TABLE 9, FIG. 3).

Odorless microorganisms were selected as dominant microorganisms which were grown in the evaporator core, created colonies during culture and did not generate an offensive odor and the culture method will be given below:

\textcircled{1} Purely isolated and cultured odorless microorganisms were inoculated in R2A liquid medium.

\textcircled{2} The inoculated medium was cultured at a temperature of 28° C. for 5 to 7 days.

\textcircled{3} An aluminum fin which had been sterilized at a high pressure at a temperature of 121° C. for 20 minutes was prepared.

\textcircled{4} The fin was immersed in each antibacterial agent to evenly coat the surface of the fin.

\textcircled{5} The coated aluminum fin was placed on a petri dish.

\textcircled{6} 1 ml of the cultured odorless microorganism inoculation solution was centrifuged and the supernatant was removed.

\textcircled{7} 1 ml of the sterilized 1×PBS was added and centrifugation was conducted again.

\textcircled{8} The method of \textcircled{7} was repeated twice.

\textcircled{9} 100 μl of the odorless microorganism washed with PBS 100 μl was dropped in the middle of the aluminum fin.

\textcircled{10} The prepared aluminum fin was inoculated with microorganisms and dried at room temperature.

\textcircled{11} The petri dish was sealed and at a temperature of 28° C. for one month.

As a result, all the combinations of the following TABLE 9 (result of odor analysis upon culture of microorganisms grown in evaporator core from which odorous microorganisms are removed) did not generate odors after one month.

TABLE 9

| | | Odor evaluation |
|---|---|---|
| Combination | Microorganism | after one month |
| 1 | Methylobacterium brachiatum | odorless |
| 2 | Methylobacterium platani | odorless |
| 3 | Methylobacterium aquaticum + | odorless |
| | Methylobacterium platani | |
| 4 | Methylobacterium platani + | odorless |
| | Methylobacterium brachiatum | |
| 5 | Methylobacterium aquaticum + | odorless |
| | Methylobacterium platani + | |
| | Methylobacterium brachiatum | |
| 6 | Methylobacterium aquaticum + | odorless |
| | Methylobacterium platani + | |
| | Methylobacterium brachiatum + | |
| | Acinetobacter johnsonii | |
| 7 | Methylobacterium aquaticum + | odorless |
| | Methylobacterium platani + | |
| | Methylobacterium brachiatum + | |
| | Bacillus vietnamensis | |
| 8 | Methylobacterium aquaticum + | odorless |
| | Methylobacterium platani + | |
| | Methylobacterium brachiatum + | |
| | Brevibacillus invocatus | |
| 9 | Methylobacterium aquaticum + | odorless |
| | Methylobacterium platani + | |
| | Methylobacterium brachiatum + | |
| | Deinococcus ficus | |
| 10 | Methylobacterium aquaticum + | odorless |
| | Methylobacterium platani + | |
| | Methylobacterium brachiatum + | |
| | Leifsonia soli | |
| 11 | Methylobacterium aquaticum + | odorless |
| | Methylobacterium platani + | |
| | Methylobacterium brachiatum + | |
| | Methylobacterium komagatae | |
| 12 | Methylobacterium aquaticum + | odorless |
| | Methylobacterium platani + | |
| | Methylobacterium brachiatum + | |
| | Pseudomonas nitroreducens | |
| 13 | Methylobacterium aquaticum + | odorless |
| | Methylobacterium platani + | |
| | Methylobacterium brachiatum + | |
| | Sphingomonas aquatilis | |
| 14 | Sphingomonas aquatilis + | odorless |
| | Brevibacillus invocatus | |
| 15 | Leifsonia soli + | odorless |
| | Methylobacterium komagatae | |
| 16 | Acinetobacter johnsonii + | odorless |
| | Sphingomonas aquatilis + | |
| | Methylobacterium komagatae | |
| 17 | Pseudomonas nitroreducens | odorless |
| 18 | Acinetobacter johnsonii + | odorless |
| | Pseudomonas nitroreducens | |
| 19 | Brevibacillus invocatus + | odorless |
| | Acinetobacter johnsonii + | |
| | Pseudomonas nitroreducens | |
| 20 | Leifsonia soli + | odorless |
| | Pseudomonas nitroreducens | |
| 21 | Brevibacillus invocatus + | odorless |
| | Sphingomonas aquatilis + | |
| | Pseudomonas nitroreducens | |
| 22 | Acinetobacter johnsonii + | odorless |
| | Sphingomonas aquatilis + | |
| | Pseudomonas nitroreducens | |
| 23 | Methylobacterium aquaticum + | odorless |
| | Methylobacterium komagatae + | |
| | Bacillus vietnamensis + | |
| | Deinococcus ficus | |
| 24 | Methylobacterium aquaticum+ | odorless |
| | Methylobacterium komagatae + | |
| | Curtobacterium flaccumfaciens + | |
| | Deinococcus apachensis + | |
| | Bacillus subtilis subsp. subtilis | |
| 25 | Methylobacterium aquaticum + | odorless |
| | Methylobacterium komagatae + | |
| | Spirosoma linguale + | |
| | Sphingomonas dokdonensis + | |
| | Leifsoniasoli | |

As shown in the test result described above, when a combination of microorganisms not generating odors is formed by removing odor-causing microorganisms grown in an air-conditioning system by a chemical or physical method, odors generated from the air-conditioning system may be significantly removed.

Although the various exemplary embodiments of the present disclosure have been disclosed for illustrative purposes, those skilled in the art will appreciate that various modifications, additions and substitutions are possible, without departing from the scope and spirit of the disclosure as disclosed in the accompanying claims.

What is claimed:

1. A method of screening an antimicrobial agent for an air-conditioning system comprising:
(a) preparing microorganism or a culture solution thereof, wherein the microorganism generates odors in the air-conditioning system;
(b) contacting the microorganism or a culture solution thereof with a sample including the antimicrobial agent;
(c) measuring growth of the microorganism; and
(d) determining whether the sample has antibacterial activity to reduce odors in an air-conditioning system when growth of the microorganism is inhibited.

2. The method of claim 1, wherein the microorganism comprises at least one microorganism selected from the group consisting of Pelomonas puraquae, Spirosoma radiotolerans, Fibrella aestuarina, Chryseobacterium geocarposphaerae, Spirosoma linguale, and Geobacillus toebii.

3. The method according to claim 1, wherein the air-conditioning system is an air conditioner.

4. The method of claim 1, wherein the microorganism forms a biofilm in an evaporator core in the air-conditioning system to induce odors.

5. The method of claim 4, wherein a material for the evaporator core comprises aluminum, an aluminum alloy, copper or a copper alloy.

6. The method of claim 1, wherein the Pelomonas puraquae is Pelomonas puraquae HKMC-113 (accession number: KCCM11689P), the Spirosoma radiotolerans is Spirosoma radiotolerans HKMC-114 (accession number: KCCM11690P), the Fibrella aestuarina is Fibrella aestuarina HKMC-115 (accession number: KCCM11691P), the Chryseobacterium geocarposphaerae is Chryseobacterium geocarposphaerae HKMC-116 (accession number: KCCM11692P), the Spirosoma linguale is Spirosoma linguale HKMC-117 (accession number: KCCM11693P), and the Geobacillus toebii is Geobacillus toebii HKMC-118 (accession number: KCCM11694P).

7. An antimicrobial agent screened by a method of claim 1.

8. A kit comprising an antimicrobial agent of claim 7.

9. A method of inhibiting growth of an odor-generating microorganism in an air-conditioning system comprising coating or spraying an antibacterial agent screened by a method of claim 1 on the air-conditioning system.

10. A method of removing odors in an air-conditioning system comprising coating or spraying an antibacterial agent to reduce odors screened by the method of claim 1 on the air-conditioning system.

11. A method of removing odors in an air-conditioning system comprising, from the air-conditioning system, isolating or removing at least one odor-generating microorganism selected from the group consisting of Pelomonas puraquae, Spirosoma radiotolerans, Fibrella aestuarina, Chryseobacterium geocarposphaerae, Spirosoma linguale, and Geobacillus toebii.

12. A method of removing odors in an air-conditioning system comprising, from the air-conditioning system, inhibiting growth of at least one odor-causing microorganism selected from the group consisting of Pelomonas puraquae, Spirosoma radiotolerans, Fibrella aestuarina, Chryseobacterium geocarposphaerae, Spirosoma linguale, and Geobacillus toebii.


SEQUENCE LISTING (1 sequence)

SEQ ID NO: 1 - DNA, 22 bp, Artificial Sequence
  PRIMER
ctacggcaaggcgacgctgacg